(19) United States
(12) Patent Application Publication
(10) Pub. No.: US 2024/0023857 A1
(43) Pub. Date: Jan. 25, 2024

(54) System and Method for Recognizing Emotions

(71) Applicant: Siemens Aktiengesellschaft, München (DE)

(72) Inventor: Rebecca Johnson, München (US)

(73) Assignee: Siemens Aktiengesellschaft, München (AT)

(21) Appl. No.: 18/042,399

(22) PCT Filed: Aug. 24, 2021
(86) PCT No.: PCT/EP2021/073311

§ 371 Date: Feb. 21, 2023

(30) Foreign Application Priority Data
Aug. 25, 2020 (DE) 10 2020 210 748.3

(51) Int. Cl. A61B 5/16; G10L 25/63; G06V 40/16; A61B 5/384; A61B 5/0205; A61B 5/00

(52) U.S. Cl. CPC A61B 5/165; G10L 25/63; G06V 40/174; A61B 5/384; A61B 5/0205; A61B 5/681; A61B 5/163; H04N 23/698

(57) ABSTRACT

Various embodiments of the teachings herein include a method for recognizing the emotional tendency of a user recorded over a defined period by two or more recording and/or capture devices. An example method comprises: generating primary data relating to the user for each device; forwarding the primary data to a server; combining the primary data in the server to form respective primary data sets for each device; assigning each primary data set individually to one or more primarily determined emotional tendencies of the user; generating secondary data by logically comparing the primarily determined emotional tendencies which have occurred at the same time; and generating a result in the form of one or more secondary emotional tendencies of the recorded and/or captured user by processing the secondary data.

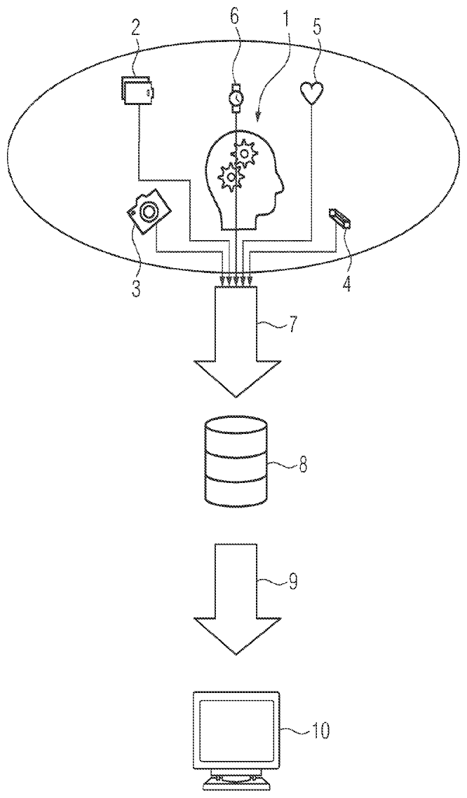

### CROSS-REFERENCE TO RELATED APPLICATIONS

This application is a U.S. National Stage Application of International Application No. PCT/EP2021/073311 filed Aug. 24, 2021, which designates the United States of America, and claims priority to DE Application No. 10 2020 210 748.3 filed Aug. 25, 2020, the contents of which are hereby incorporated by reference in their entirety.

### TECHNICAL FIELD

The present disclosure relates to video communication. Various embodiments of the teachings herein include systems and/or methods for recognizing emotions of a user within a defined period, e.g., which can be used both in a mobile manner—that is to say in situ—and via a screen.

### BACKGROUND

It is known that a statement, whether communicated in writing, orally and/or optically, can be assigned to an emotional basic tendency such as “relaxed”, “cheerful”, “aggressive” or “anxious”. For a wide variety of data collections, for example also for optimally designing a workstation in a factory, it is useful to know the conscious and/or unconscious reactions of a user to the environment so that it can be optimized in an individualized manner.

There are already a number of methods and systems for recognizing emotions. A particular emotional basic tendency can therefore be assigned to the author of a text, whether the author is communicated in writing and/or orally, at the time at which the text is created by finding various keywords, for example “laugh, fun, wit, joy” etc., within the text. Although this technique for recognizing emotional basic tendencies already works, it is not yet fully developed because typical human behaviors, for example irony, are often not recognized and/or are misinterpreted. For example, the expression “this will be fun!”, which is easily recognized by a person as ironic, would probably be incorrectly assigned using the existing technique. In addition, “laughing” per se cannot be readily identified and is sometimes assigned completely incorrectly, for example as “screaming”.

On the other hand, emotional tendencies can also be captured by means of biometric body and/or facial recognition, wherein an appropriately equipped system can carry out the assignment in an automated manner by recognizing stored facial features such as frown lines, laughter lines, upturned corners of the mouth, showing one's teeth etc. This is particularly important because facial recognition, in particular, is a strong indicator of emotions. If we laugh or cry, we allow the environment to look into our innermost being and to react accordingly. However, much less pronounced expressions also reveal emotions which can be used beneficially and are therefore worthy of being recognized in an automated manner.

Although there are methods for recognizing emotions which use facial recognition from Google, Amazon and Microsoft, these methods for recognizing emotions are not yet fully developed. For example, a facial recognition system established in Russia recognizes all Asian faces as “in a good mood” or “happy” because their eye folds are curved in a particular way. The same applies to optical data—for example video recordings—of users that are classified as “angry”, said users simply exhibiting wrinkles as a result of aging and not because of their current state of mind.

As a result of the advancing automation, there is the need to provide a method for recognizing emotions which at least partially avoids the errors of the existing techniques for recognizing emotions.

### SUMMARY

The teachings of the present disclosure provide systems and/or methods for recognizing emotions—e.g., in an automated manner—which overcome the disadvantages of the prior art. For example, some embodiments include a method for recognizing the emotional tendency of a user (1) recorded over a defined period by two or more recording and/or capture devices (2, 3, 4, 5, 6), said method comprising: generating primary data relating to the user for each recording and/or capture device, forwarding (7) the primary data to a server (8), combining the primary data in the server (8) to form respective primary data sets for each recording and/or capture device (2, 3, 4, 5, 6) by processing the primary data, assigning each primary data set individually and in a computer-aided manner, preferably automatically, to one or more primarily determined emotional tendencies of the user (1), generating secondary data by logically comparing the primarily determined emotional tendencies which have occurred at the same time in a computer-aided manner and/or automatically, and generating a result in the form of one or more secondary emotional tendencies of the recorded and/or captured user (1) by processing the secondary data.

In some embodiments, at least three recording and/or capture devices (2, 3, 4, 5, 6) are used at the same time.

In some embodiments, audio data relating to the user (1) are generated as primary data.

In some embodiments, video data relating to the user (1) are generated as primary data.

In some embodiments, electroencephalography results for the user (1) are collected as primary data.

In some embodiments, heart rate data relating to the user (1) are collected as primary data.

In some embodiments, speech or text analysis data are collected as primary data.

As another example, some embodiments include a system for recognizing the emotional tendency of a user (1) recorded and/or captured by a sensor system, said system at least comprising the following modules: at least two devices (2, 3, 4, 5, 6) for recording and/or capturing primary data relating to the user (1), appropriate means (7) for passing the primary data generated in this manner to a server (8), the server (8) which processes the primary data, a connection (9) between the server (8) and an output device (10), and the output device (10) for outputting the result of the computer-aided processing of the secondary data in the form of a report relating to one or more secondary emotional tendencies of the user (1) recorded and/or captured over a defined period.

In some embodiments, the recording and/or capture device is an input means of a computer.

In some embodiments, recording and/or capture device is a camera.

In some embodiments, a recording and/or capture device comprises 360° camera technology.

In some embodiments, a recording and/or capture device comprises an electroencephalograph—EEG.

In some embodiments, a recording and/or capture device comprises a smartwatch.

In some embodiments, a recording and/or capture device comprises a gaze detection apparatus.

In some embodiments, at least one module of the system is mobile.

### BRIEF DESCRIPTION OF THE DRAWINGS

The teachings of the present disclosure are explained below on the basis of a FIGURE which schematically shows an example of an embodiment of the system for recognizing the emotional tendency of a user recorded and/or captured by means of a sensor system.

### DETAILED DESCRIPTION

Some embodiments of the teachings herein include a method for recognizing the emotional tendency of a user recorded over a defined period by two or more recording and/or capture devices. An example method includes:
generating primary data relating to the user for each recording and/or capture device,
forwarding the primary data to a server,
combining the primary data in the server to form respective primary data sets for each recording and/or capture device by processing the primary data,
assigning each primary data set individually and in a computer-aided manner, preferably in an automated manner, to one or more primarily determined emotional tendencies of the user,
generating secondary data by logically comparing the primarily determined emotional tendencies which have occurred at the same time in a computer-aided and/or automated manner,
generating a result in the form of one or more secondary emotional tendencies of the recorded and/or captured user by processing the secondary data.

Some embodiments include a system for recognizing the emotional tendency of a user recorded and/or captured by a sensor system. An example system may include: at least two devices for recording and/or capturing primary data relating to the user, appropriate means for passing the primary data generated in this manner to a server, the server which processes the primary data, a connection between the server and an output device, and the output device for outputting the result of the computer-aided processing of the secondary data in the form of a report relating to one or more secondary emotional tendencies of the user recorded and/or captured over a defined period.

In some embodiments, a system comprises the following modules, for example:
two or more recording and/or capture devices for generating the primary data,
a line, in particular to a server,
a server which receives, stores and processes the primary data and generates, transmits, stores and/or processes secondary data,
a line from the server to a readout device,
a readout device.

Since all of these modules can be easily obtained in versions in which they fit into a briefcase and/or a suitcase, the entire system may be mobile and may be offered as transportable.

On the other hand, individual, all or a plurality of the modules may be mounted in a stationary and fixed manner, wherein the output device may be designed to be mobile, for example, and the capture device may be designed to be stationary or vice versa.

An effective system for recognizing the emotional tendency comprises a plurality of capture and/or recording devices which simultaneously recognize, for example even in real time, the emotions of the user from various viewing angles, that is to say, for example, optically, acoustically—based on the volume of the sounds—, from the spoken word and/or from the gestures, the posture or the facial expression. These data are then collected, based on the time and based on a user, and are processed by means of artificial intelligence—AI. The AI can then not only validate the correctness of the individual results by means of cross-checks, but can also recognize patterns. If a gesture, in particular also an involuntary gesture, for example raising of eyebrows, recurs often enough, the AI will assign this a result regarding the emotion linked thereto—verified by the other results of the processing of the primary data. For this user, the AI is then trained, for example, to assign an emotion verified by other data, for example “skepticism”, to the raising of the eyebrows.

The methods and/or systems may be used not only to check machine-captured emotions, but rather to complete an emotion recognized—using primary data—by capturing many different signals which are consciously or unconsciously emitted by the user and represent his/her emotional state. In this case, the AI is trained in a manner personalized to the user(s).

In this disclosure, the audio data, video data and/or other data obtained by devices for capturing the state of the user before processing by the server are referred to as “primary data”.

In this disclosure, the audio data, video data and/or other data obtained by processing and/or logically comparing the primary data are referred to as “secondary data”.

In this disclosure, a group of data which are related in terms of content and have identical structures, for example the value of the heart rate assigned to a time over a certain period in each case, is referred to as a “data set”. A data set may be generated from data, processed in a computer-aided manner, stored, compared, combined with other data sets, calculated, etc. This generally happens in a server.

“Devices for capturing” the state of the user are, for example, recording devices and/or sensors which capture the speech, the facial expression, the gestures, the posture, the heart rate, the pulse, the brain waves and/or the muscle tension of the user and convert it/them into data.

Artificial intelligence (AI), for example, can be trained using the primary and/or secondary data. In this case, the assignment of primary data to (an) emotional basic tendency/tendencies can be trained in an automated and/or personalized manner in an automated manner and/or by way of an individual decision by the user in iterative optimization steps.

In some embodiments, the AI trained in a manner personalized to the user or the group of users—for example over-60s with typical wrinkles, people with slanting eyes, people with hooded eyelids and/or drooping eyelids, people with particularly pronounced eyebrows, etc.—avoids misinterpretations of invariable facial features because the forehead wrinkle which is initially captured as “angry”, for example, and can also be recognized when the user is in a great mood and in a happy mood has nothing to do with anger for this user and the AI is trained in a personalized and individualized manner.

In addition, the division, which can be classified as “racist”, into the six conventional facial expressions “angry”, “disgusted”, “anxious”, “happy”, “sad” and “surprised”, in the case of which Japanese faces are classified again and again as “happy” on account of the eye position and African faces are classified again and again as “angry”, again owing to the eye position, is dispensed with when using the methods and/or systems described herein.

When processing the primary data, the AI can then assign said data to a corresponding group of users and can correct the recognition of emotions in a manner typical of this group. The AI can also possibly assign various significances to the captured primary data, with the result that, for example, an involuntary gesture or a facial movement which cannot be deliberately controlled receives a higher significance than conventional smiling and/or the voice recognition of the sentence “I'm well”. This is because, in particular, these two machine-recognizable emotions mentioned last do not always mean “happiness”, but sometimes can be simply assigned to a good expression and do not actually represent a good mood.

This is because, in particular, the voice recognition recognizes politeness and a friendly mood, for example, if the user shows only his “facade” and is in a tense mood. It is even more extreme if irony or sarcasm is involved since a conventional system generally recognizes precisely the opposite of the emotional state of the user.

In order to recognize sarcasm or irony, the system needs a multiplicity of primary data items which decipher the true meaning of the spoken word. The method disclosed here can correctly interpret this using the many different devices for capturing the primary data, which, in addition to capturing the spoken word, each also capture statements relating to the pitch, the eye expression, the lip tension, the gestures of the hands, the posture, the body tension, the environment in which the user is situated—for example the boss is behind him/her—etc., and as a result of the fact that these primary data of “non-verbal communication” are available to the voice recognition at the same time as the primary data of “verbal communication” for processing, and can provide secondary data and results which precisely identify the sarcasm.

As a result of the user being recorded and/or captured, audio and/or video data relating to a user are captured at the same time, for example, and can then be assigned according to the question “what happened at the same time?” during the—computer-aided—logical comparison and/or generation of the secondary data: optical and/or acoustic data from two or more capture devices such as:
1) capture of biometric facial features,
2) assignment of keywords in the spoken/written text,
3) assignment
   a) of the pitch of the acoustic presentation,
   b) of the volume of the voice,
4) assignment of the head posture of the speaker when speaking particular passages in the text, and so on.

The combined data can be compared in the server in an automated manner for each interval of time, with the result that data are obtained from results which are compared per se and are therefore conclusive, said data, as secondary data, forming the basis for the secondarily determined emotional tendency at a given time.

The “recording and/or capture device” comprises, for example, one or more of the following
an input device for a computer, such as a keyboard, a mouse, a stylus, a stick,
a camera, a 3D camera, 360° camera technology,
a microphone,
an electroencephalograph “EEG”, in particular a so-called “EEG cap”,
a pulse meter, a heart rate monitor, for example in the form of a smartwatch,
a gaze detection device which captures, for example, points which are being considered closely, fast eye movements and/or other gaze movements of a user and generates primary data therefrom,
other devices with a sensor system for capturing body-specific and/or physical data relating to the user,
all of the above-mentioned devices are used in the system at least in pairs and/or in any desired combinations and also in combination with other recording and/or capture devices for capturing an overall recording of the user.

As a result of the primary data relating to the user, who will generally be a person, being recorded and/or captured over a certain period by means of the recording and/or capture device(s), visible and invisible, consciously articulated and/or unconsciously shown facial expressions and facial micro-expressions, expressions, the posture, gestures and/or measurable changes in the circulation of the user are captured over a particular period and are accordingly converted into primary data.

These primary data are passed to a computer-aided device, in particular a server. There, the primary data are stored, for example in the form of primary data sets, and/or are processed to form primary data sets. Each primary data set, to which only one recording and/or capture device can generally be assigned, is assigned a primarily captured emotional tendency, based on a respective time at which the data are captured and the generating device, by virtue of the processing in the server. This intermediate result is stored for each device as a primary data set and a primarily determined emotional tendency—in each case based on a time.

“360° camera technology” denotes when the cameras make it possible for the user to package experience in a 360° panoramic image film. This may take place in augmented reality, virtual reality and/or mixed reality. The viewer is provided with a sense of being close to the event. 360° cameras are available on the market. 360° camera recordings can also be mixed with virtual elements. In some embodiments, elements may be highlighted by means of markings, for example. This is a common technique, for example in football reports.

A 360° 3D camera has, for example, a certain number of lenses installed in the 3D camera. 3D cameras having only one lens may cover 360° using the fisheye principle and at least may film at an angle of 360°×235°. The digital data generated by the 3D cameras in the room for recording are transmitted to one or more servers. Here the system may recognize, for example, who is behind the user or who is behind the 2D camera capturing the user.

A computer program and/or a device which very generally provides functionalities for other programs and/or devices is referred to as a “server”. A hardware server is a computer on which one or more “servers” run.

In some embodiments, all primary data are transmitted to one or more servers. The “server” initially assigns these data to primary emotional tendencies, then processes them to form secondary data and assigns the latter to (a) secondary emotional tendency/tendencies in a computer-aided manner. The server transmits and/or passes the result of this calculation to an output device.

Unless stated otherwise in the following description, the terms “process”, “carry out”, “produce”, “computer-aided”, “calculate”, “transmit”, “generate” and the like preferably relate to actions and/or processes and/or processing steps which change and/or generate data and/or convert the data into other data, in which case the data may be represented or may be present, in particular, as physical variables, for example as electrical pulses.

The expression “server” should be interpreted as broadly as possible so as to cover all electronic devices having data processing properties, in particular. Servers may therefore be, for example, personal computers, handheld computer systems, pocket PC devices, mobile radio devices and other communication devices which can process data in a computer-aided manner, processors and other electronic data processing devices.

In this disclosure, “computer-aided” may be understood as meaning, for example, an implementation of the method in which a server, in particular, carries out at least one method step of the method using a processor.

All primarily captured emotional tendencies are calculated as the processing result in the server. They are then available as data and form the data basis for generating the secondary data and/or secondary data sets and the resulting secondary emotional tendency at the respective time, which is ultimately forwarded to the output device.

Moods and feelings which are expressed via the captured primary data are referred to as an “emotional tendency”. For example, smiling in combination with wide open eyes and a raised head are signs of a good mood, self-confidence, etc. There are likewise combinations which are indicators of anxiety, rage, pain, sadness, surprise, calm, relaxation, disgust etc.

Logical and computer-aided processing of the primarily captured emotional tendencies generates secondary data which reveal a secondary or resulting emotional tendency of the respective user at the respective time. As a result of the combinational consideration of all available primary data, irony, sarcasm, aging wrinkles etc., for example, can be assigned correctly or at least in a considerably improved manner than in the case of individual consideration of the primary data, as is the prior art.

The secondary data can also be used to identify, delete and/or reject implausible data in the primary data set(s). For example, this may be carried out in an individualized manner by way of a decision by the user or in an automated manner using appropriately trained artificial intelligence.

Finally, the secondary data and/or the secondary data sets, for example, are based only on primary data relating to the user which make sense during the combined consideration of all primary data within the scope of the resulting secondary data set. Primary data which in that respect “do not fit into the image” are identified, for example, during the processing of primary data sets to form secondary data and are separately assessed, rejected and/or deleted.

Appropriate processing of the secondary data produces—in each case based on the same time—the secondary emotional tendency which is the result of the examination. A resulting overall result is then generated from the secondary data using an algorithm and is made visible using the output device.

The secondary and therefore comparatively clearly and correctly interpreted emotional tendencies of the user in the respective situation can be used to draw conclusions which make it possible to optimize all locations and environments in which people are located. For example, workstations can be optimized, a factory process can be optimized, an interior of a vehicle, such as a train, an automobile etc., can be optimized.

Recurring gestures and patterns, combinations and relationships can then be recognized in an automated manner, for example, using artificial intelligence and can be deliberately searched for within the period in question. These allow the user to draw conclusions on the emotional effect of a particular company, environment, situation, color, daylight.

The user can also draw conclusions therefrom which are possibly not known to the user such that, for example, when reaching into the shelf in a particular manner or during the associated rotating movement of the wrist, the user always painfully moves his face. If the user pushes the screw box somewhat to the left, the user avoids the pain which he/she would not have been made aware of at all without a tool such as the method and system proposed here for the first time.

In some embodiments, the assignments of the primary data are corrected in a personalized manner by an individual user, with the result that artificial intelligence can be trained thereby, for example, and then in turn modifies the rules for assigning the primary data in a personalized manner. For example, the method and the system can then learn to distinguish the well-intentioned smiling of a person from the derisive smirking of the same person.

A user-trained system with pattern recognition is therefore disclosed here for the first time and provides solutions of the captured primary data which are matched specifically to the user in a personalized manner and recognize, for example, a poker face as what it is and not as what would be interpreted by conventional facial recognition. For example, the user can then also query in an automated manner the situation in which the user was particularly relaxed, happy and/or satisfied.

The term “automatic system” or “automatic” or “automated” here represents an automatic, in particular computer-aided automatic, sequence of one or more technical processes according to a code, a defined plan and/or with respect to defined states. The range of automated sequences is as great as the possibilities, the computer-aided processing of data per se.

A monitor, a handheld, an iPad, a smartphone, a printer, a voice output, etc. is used as an “output device”, for example. Depending on the output device, the form of the “report” may be a printout, a display, a voice output, a pop-up window, an email or other ways of reproducing a result.

The primary data, for example the audio and video data of a film recording of a user in a situation over a defined period, can naturally also be directly followed and made available via playback devices. On account of the automated processing of the primary data to form secondary data, it is also possible to deliberately manually start a search for patterns based on a person and/or a situation. In some embodiments, the data sets which are used to train the AI are generated as results that have already been compared according to the method defined further above.

The already available methods for recognizing the emotional basic tendencies of a user each have error sources per se, but these error sources can be minimized by comparing the results of different recognition methods with one another. In addition, it is general knowledge of the present invention that the error sources can be avoided in a personalized manner by virtue of the individual user training his/her device to his/her emotional expressions. In a further example, the AI can then develop enhanced recognition methods on the basis of the training. Ultimately, AI can then assign a user to a particular cluster, in which case the emotions of the users in similar “clusters” can then be recognized in a more correct manner even without personalized training of a system.

The FIGURE shows the head of a user 1 who is active. The user's conscious and unconscious utterances are captured by means of a video camera 2, a 360° camera 3, a microphone 4, and a heart rate monitor 5, for example in the form of a smartwatch 6. These devices each individually forward primary data to a server 8 via the data line 7. In the server, primary emotional tendencies are first of all calculated from these primary data and are then compared. Finally, the server 8 calculates the secondary emotional tendencies during the period in question from the secondary data. These results are forwarded to an output device 10 via the data line 9.

For example, if emotional text is recognized by the microphone 4 on the basis of the keywords, but the video camera 2 records rather angry facial features for facial recognition and the voice recognition finally recognizes a loud and rather angry voice via the microphone 4, the system can assign “sarcasm” as a secondarily recognized emotional tendency by virtue of processing in the server 8.

Various embodiments of the teachings herein include methods and systems for recognizing the emotions of a user, in which the individual results in the form of the primary emotional tendencies together and in their combination produce a resulting, so-called “secondarily calculated”, emotional tendency which is assessed as the result of the examination. Not only are a wide variety of methods of the sensor system combined in this case, but rather they are possibly also individually trained, that is to say assigned and/or interpreted manually or in an automated manner, in which case their relevance with respect to the individual user is evaluated. A corresponding user profile can thus be created.

What is claimed is:

1. A method for recognizing the emotional tendency of a user recorded over a defined period by two or more recording and/or capture devices, the method comprising:
generating primary data relating to the user for each recording and/or capture device;
forwarding the primary data to a server;
combining the primary data in the server to form respective primary data sets for each recording and/or capture device by processing the primary data;
assigning each primary data set individually and in a computer-aided manner to one or more primarily determined emotional tendencies of the user;
generating secondary data by logically comparing the primarily determined emotional tendencies which have occurred at the same time in a computer-aided manner and/or automatically; and
generating a result in the form of one or more secondary emotional tendencies of the recorded and/or captured user by processing the secondary data.

2. The method as claimed in claim 1, wherein there are at least three recording and/or capture devices.

3. The method as claimed in claim 1, wherein the primary data comprises audio data relating to the user.

4. The method as claimed in claim 1, wherein the primary data comprises video data relating to the user.

5. The method as claimed in claim 1, wherein the primary data comprises electroencephalography results for the user.

6. The method as claimed in claim 1, wherein the primary data comprises heart rate data relating to the user.

7. The method as claimed in claim 1, wherein the primary data comprises speech or text analysis data.

8. A system for recognizing the emotional tendency of a user recorded and/or captured by a sensor system, the system comprising:
at least two devices for recording and/or capturing primary data relating to the user;
a server;
a transmitter for passing the primary data generated in this manner to the server;
wherein the server processes the primary data to generate secondary data;
an output device communicating a result of the computer-aided processing of the secondary data in the form of a report relating to one or more secondary emotional tendencies of the user recorded and/or captured over a defined period.

9. The system as claimed in claim 8, wherein at least one recording and/or capture device comprises an input of a computer.

10. The system as claimed in claim 8, wherein at least one recording and/or capture device comprises a camera.

11. The system as claimed in claim 8, wherein at least one recording and/or capture device comprises 360° camera technology.

12. The system as claimed in claim 8, wherein at least one recording and/or capture device comprises an electroencephalograph.

13. The system as claimed in claim 8, wherein at least one recording and/or capture device comprises a smartwatch.

14. The system as claimed in claim 8, wherein at least one recording and/or capture device comprises a gaze detection apparatus.

15. The system as claimed in wherein at least one module of the system is mobile.
